(19) United States
(12) Patent Application Publication
(10) Pub. No.: US 2024/0242538 A1
(43) Pub. Date: Jul. 18, 2024

(54) SYSTEMS FOR CONTROLLING MEDIA PLAYBACK

(71) Applicant: David K. Pederson, Mulino, OR (US)

(72) Inventor: David K. Pederson, Mulino, OR (US)

(21) Appl. No.: 18/587,630

(22) Filed: Feb. 26, 2024

Related U.S. Application Data
(63) Continuation-in-part of application No. 18/082,866, filed on Dec. 16, 2022, now Pat. No. 11,989,974.
Application No. 18/082,866 is a continuation of application No. 16/859,727, filed on Apr. 27, 2020, now Pat. No. 11,568,680.

(51) Int. Cl. G06V 40/16; A61B 5/00; A61B 5/021; A61B 5/16

(52) U.S. Cl. CPC G06V 40/174; A61B 5/0022; A61B 5/0077; A61B 5/021; A61B 5/165; A61B 5/4848; A61B 5/681; A61B 5/6898; G06V 40/166; G06V 40/172

(57) ABSTRACT

Systems for controlling media playback based on a person exhibiting a smile with therapeutic benefits. The systems include a facial expression detection device, a system processor, and a media device. The system processor is in data communication with the facial expression detection device and is configured to execute stored computer executable system instructions. The media device is controllably coupled to the system processor and configured to play and stop playing a media file in response to playback instructions from the system processor. The computer executable system instructions include prompting the person to exhibit a smile with therapeutic benefits, receiving facial expression parameter data, receiving current facial expression data, comparing the current facial expression data to the facial expression parameter data, identifying whether the current facial expression data satisfies target facial expression criteria, and sending playback instructions to the media device based on the facial expression data satisfaction identification.

### CROSS REFERENCE TO RELATED APPLICATIONS

This application claims priority to copending U.S. patent application Ser. No. 18/082,866, filed on Dec. 16, 2022, which is a continuation of U.S. Pat. No. 11,568,680, issued on Jan. 31, 2023; and which are hereby incorporated by reference in their entirety for all purposes.

### BACKGROUND

Smiling is known to non-verbally communicate emotions, like happiness and joy, and other messages, such as approval between people. Lesser known is the fact that smiling can provide health benefits, both to the person observing another smiling and to the person smiling. This document will focus on the therapeutic health benefits from smiling to the person who is smiling.

Genuine smiling, often called a Duchenne smile, is a particular manner of smiling that has distinct health benefits. A genuine smile improves mood, reduces blood pressure, reduces stress, reduces pain, strengthens the immune system, strengthens relationships, increases attractiveness, and improves longevity. A genuine smile is characterized by activating muscles near the eyes and cheeks in contrast to a fake or perfunctory smile that merely involves shaping the lips.

Conventional facial recognition systems are capable of detecting certain expressions on a person's face, but are not designed to detect genuine smiles. Further, existing facial recognition systems do not include features to train people how to execute a genuine smile. Conventional facial recognition systems also lack features to encourage people to execute a genuine smile with a given frequency, for a given amount of time, and/or in response to a physiological trigger.

Thus, there exists a need for smile detection systems that improve upon and advance the design of known facial recognition systems. Examples of new and useful smile detection systems relevant to the needs existing in the field are discussed below.

### SUMMARY

The present disclosure is directed to systems for controlling media playback based on a person exhibiting a smile with therapeutic benefits. The systems include a facial expression detection device, a system processor, and a media device.

The system processor is in data communication with the facial expression detection device and is configured to execute stored computer executable system instructions. The media device is controllably coupled to the system processor and configured to play and stop playing a media file in response to playback instructions from the system processor.

The computer executable system instructions include prompting the person to exhibit a smile with therapeutic benefits, receiving facial expression parameter data, receiving current facial expression data, comparing the current facial expression data to the facial expression parameter data, identifying whether the current facial expression data satisfies target facial expression criteria, and sending playback instructions to the media device based on the facial expression data satisfaction identification.

### BRIEF DESCRIPTION OF THE DRAWINGS

FIG. 1 is a perspective view of a system for detecting when a person exhibits a smile with therapeutic benefits incorporated into a watch on a person's wrist.

FIG. 2 is a schematic view of the system shown in FIG. 1.

FIG. 3 is a flow diagram of computer executable instruction steps that the system shown in FIG. 1 is programmed to follow.

FIG. 4 is a flow diagram showing additional computer executable instruction steps associated with the step of prompting the person to exhibit a facial expression.

FIG. 5 is a flow diagram showing alternative additional computer executable instruction steps associated with the step of prompting the person to exhibit a facial expression.

FIG. 6 is front view of a second example of a system for detecting when a person exhibits a smile with therapeutic benefits, the system incorporated into a smart phone.

FIG. 7 is a perspective view of a third example of a system for detecting when a person exhibits a smile with therapeutic benefits incorporated into a computer with a camera.

FIG. 8 is a is a schematic view of a system for controlling media playback.

FIG. 9 is a flow diagram of computer executable instructions utilized by the system shown in FIG. 8.

FIG. 10 is a flow diagram of a computer executable instruction included in the instruction of prompting a person to exhibit a smile shown in FIG. 9.

FIG. 11 is a flow diagram of a computer executable instructions included in the instructions shown in FIG. 9.

FIG. 12 is a flow diagram of a computer executable instructions included in the instructions shown in FIG. 9.

FIG. 13 is a flow diagram of alternative computer executable instructions that may be utilized by a system for controlling media playback.

FIG. 14 is a flow diagram of a computer executable instructions included in the instructions shown in FIG. 13.

FIG. 15 is a flow diagram of alternative computer executable instructions that may be utilized by a system for controlling media playback.

FIG. 16 is a flow diagram of alternative computer executable instructions that may be utilized by a system for controlling media playback.

### DETAILED DESCRIPTION

The disclosed smile detection systems will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various smile detection systems are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

### Therapeutic Smile Detection Systems

With reference to the figures, therapeutic smile detection systems will now be described. The systems discussed herein function to detect when a user is executing a genuine smile, also known as a Duchenne smile. The systems described in this document also function to train a user to execute a genuine smile. Another function of the systems described herein is to encourage people to execute a genuine smile to promote associated therapeutic benefits and to help establish healthy habits.

The reader will appreciate from the figures and description below that the presently disclosed systems address many of the shortcomings of conventional smile detection systems. For example, the systems described herein are sophisticated enough to detect genuine smiles in contrast to conventional facial recognition systems, which can detect only certain general expressions on a person's face. Further, the presently disclosed systems train people how to execute a genuine smile to enable them to experience the health benefits of genuine smiles. The systems discussed in this document improve over conventional facial recognition systems by encouraging people to execute a genuine smile with a given frequency, for a given amount of time, and or in response to a physiological trigger.

### Contextual Details

Ancillary features relevant to the smile detections described herein will first be described to provide context and to aid the discussion of the smile detection systems.

### Person

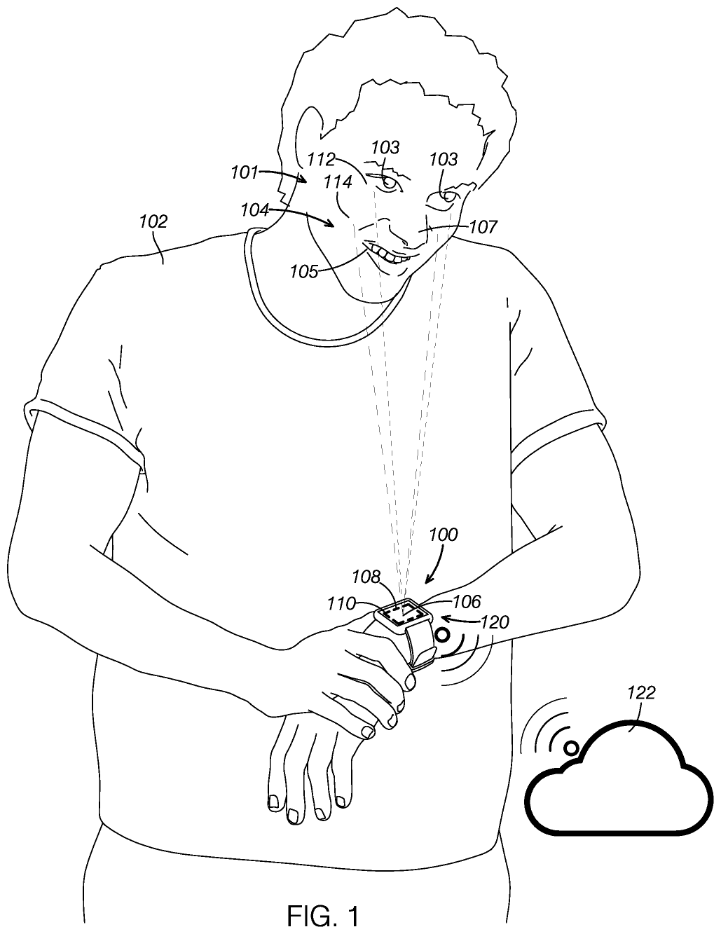

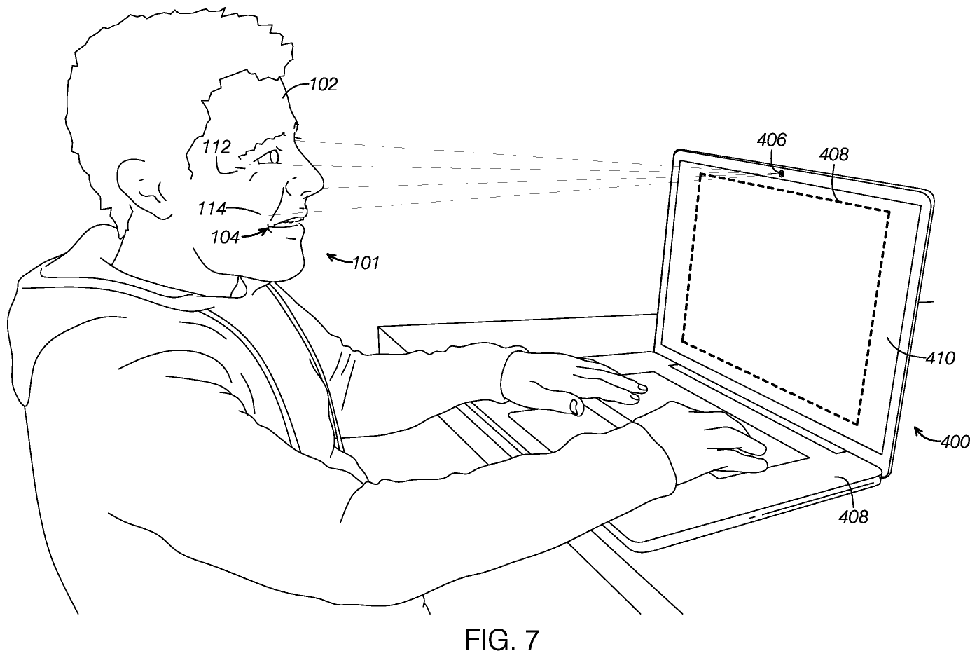

The smile detection systems described herein function to detect a smile and other facial expressions of a person, which may also be referred to as a user. With reference to FIGS. 1 and 7, a person 102 is depicted using smile detection system 100 in FIG. 1 and using smile detection system 400 in FIG. 7. Person 102 includes a face 101, eyes 103, a mouth 105, and a nose 107. Further, person 102 includes orbicularis oculi muscles 112 near eyes 103 and zygomatic major muscles 114 near mouth 105. As shown in FIGS. 1 and 7, person 102 may exhibit a smile 104.

### Smile Detection System Embodiment One

With reference to FIGS. 1-5, a first example of a smile detection system, smile detection system 100, will now be described. Smile detection system 100 includes a facial expression detection device 106, a system processor 108, and a display 110. In some examples, the smile detection system does not include one or more features included in smile detection system 100. For example, some smile detection system examples do not include a display. In other examples, the smile detection system includes additional or alternative features.

### Facial Expression Detection Device

Facial expression detection device 106 is configured to acquire facial expression data. The facial expression data may include information about the position of facial features, such as the eyes 103, nose 107, mouth 105, and ears of person 102. The facial expression data may be more granular, such as the position of specific facial muscles, such as the zygomatic major muscles 114 and/or the orbicularis oculi muscles 112 of person 102. The facial expression data may include information related to how facial features have moved by comparing the position of a given feature over time.

Additionally or alternatively to information about particular facial features, the facial expression data may include information about expressions the person is exhibiting. The expression a person is exhibiting may be determined by combining information about facial features and/or by associating expressions with defined indicators. For example, wrinkles near a person's eyes or upturned lips may be defined as indicators for a smile.

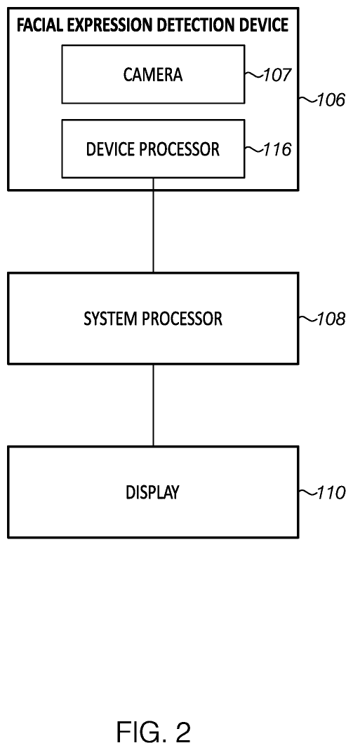

As can be seen in FIGS. 1 and 2, facial expression detection device 106 includes a camera 107 and a device processor 116. Camera 107 is configured to collect facial expression data from person 102.

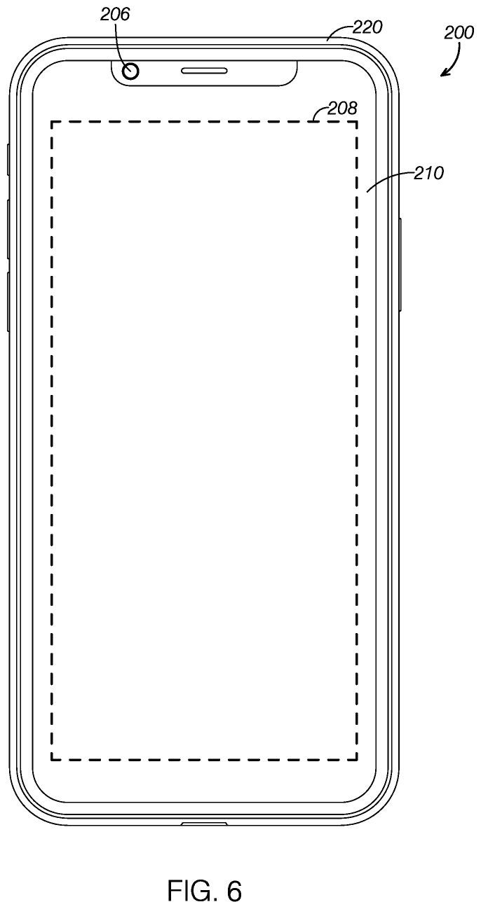

The camera may be any currently know or later developed camera suitable for collecting facial expression data from a person. In the example shown in FIG. 1, camera 107 is incorporated into a watch 120. In the example shown in FIG. 6, the camera is incorporated into a smartphone 220. In the example shown in FIG. 7, the camera is incorporated into a laptop computer 420.

Device processor 116 is configured to execute stored computer executable facial recognition instructions. The device processor may be any currently known or later developed processor suitable for executing computer executable instructions. The facial recognition instructions may be customized for detecting smiles with the facial expression detection device or may be more generally applicable facial recognition instructions.

As can be seen in FIG. 1, facial expression detection device 106 is incorporated into a watch 120. In particular, watch 120 is a smart watch with various computing features. However, the watch may be a traditional watch without computing features beyond the facial expression detection device. FIG. 6 depicts an example where a facial expression detection device 206 is incorporated into a handheld computing device in the form of a smartphone 220. FIG. 7 depicts an example where a facial expression detection device 406 is incorporated into a personal computing device in the form of a laptop computer 420.

### System Processor

System processor 108 is configured to execute stored computer executable system instructions 300. As shown in FIG. 2, system processor is in data communication with facial expression detection device 106 and with display 110.

The system processor may be any currently known or later developed processor suitable for executing computer executable instructions. The system instructions may include instructions customized for detecting a smile 104 with facial expression detection device 106, such as system instructions 300, and more generally applicable facial recognition instructions.

### Computer Executable System Instructions

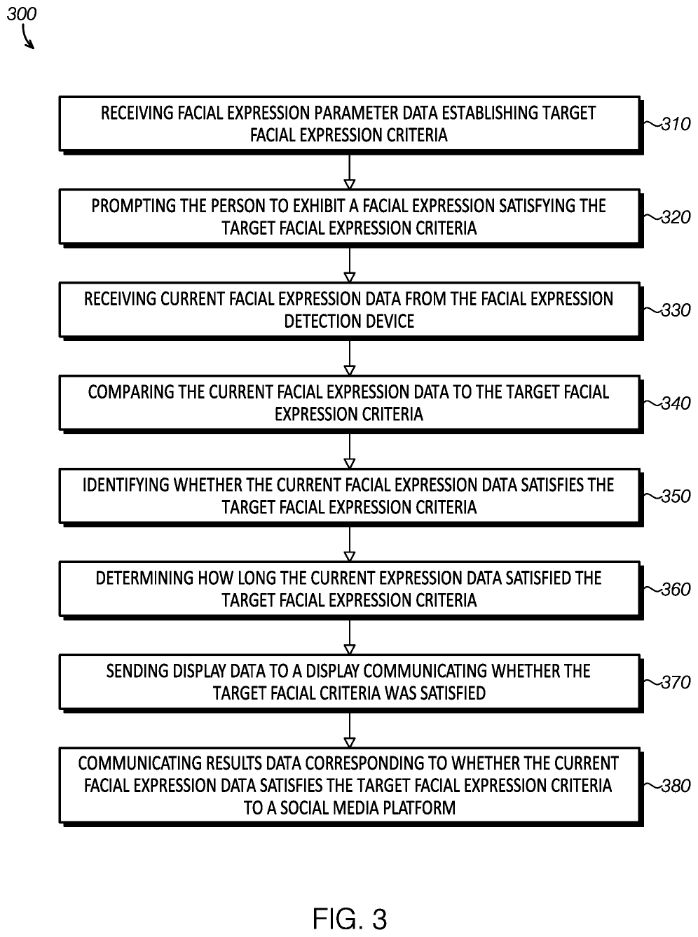

With reference to FIGS. 3-5, a particular set of computer executable system instructions, system instructions 300, will be described. The reader should appreciate that additional or alternative system instructions may be used in different examples.

As shown in FIG. 3, system instructions 300 include the step of receiving facial expression parameter data establishing target facial expression criteria at step 310. The target facial expression criteria define a smile with therapeutic benefits. Examples of smiles with therapeutic benefits include a genuine smile, which is also known as a Duchenne smile.

The target facial expression criteria may define a smile with therapeutic benefits as occurring when zygomatic major muscles 114 of person 102 contract to a selected extent. Additionally or alternatively, the target facial expression criteria may define a smile with therapeutic benefits as occurring when orbicularis oculi muscles 112 of person 102 contract to a selected extent.

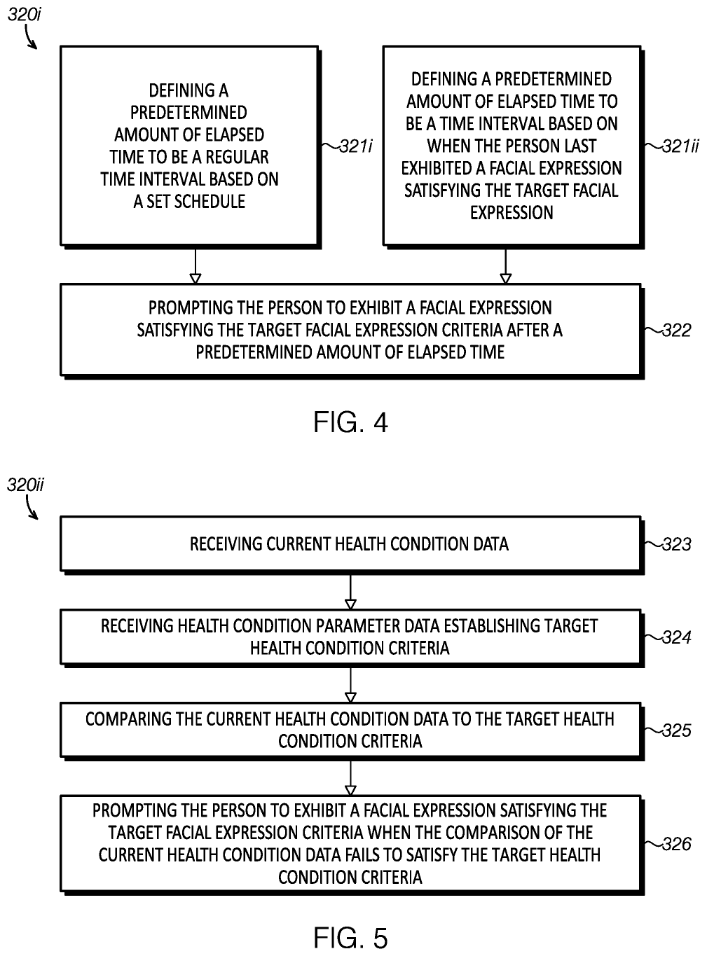

As can be seen in FIG. 3, system instructions 300 include prompting person 102 to exhibit a facial expression satisfying the target facial expression criteria at step 320. FIG. 4 depicts one example of prompting a person to exhibit a facial expression at step 320i. FIG. 5 depicts another example of prompting a person to exhibit a facial expression at step 320ii.

Prompting a person to exhibit a facial expression at step 320 may occur after a predetermined amount of elapsed time, such as shown at steps 321i and 321ii of step variation 320i in FIG. 4. Additionally or alternatively, prompting a person to exhibit a facial expression at step 320 may occur when a comparison of current health condition data fails to satisfy target health condition criteria, such as shown at step 326 of step variation 320ii in FIG. 5.

With reference to FIG. 4, the reader can see additional steps involved with prompting person 102 to exhibit a facial expression satisfying the target facial expression criteria in step variation 320i. Step 321i includes defining a predetermined amount of elapsed time to be a regular time interval based on a set schedule. Additionally or alternatively to step 321i, step 321ii includes defining a predetermined amount of elapsed time to be a time interval based on when person 102 last exhibited a facial expression satisfying the target facial criteria. Step variation 320i includes prompting person 102 to exhibit a facial expression satisfying the target facial expression criteria after a predetermined amount of elapsed time at step 322.

With reference to FIG. 5, the reader can see that prompting person 102 to exhibit a facial expression satisfying the target facial expression criteria in step variation 320ii includes receiving current health condition data at step 323. In the step 320iiexample, the current health condition data corresponds to the health of person. The current health condition data may include blood pressure data, body temperature data, pulse rate data, metabolic data, and various other types of physiological data. In certain examples, the current health condition data includes the current facial expression data, such as whether person 102 is smiling, scowling, frowning, or tensing facial muscles.

At step 324, prompting a person to exhibit a facial expression at step 320iifurther includes receiving health condition parameter data establishing target health condition criteria. The target health condition criteria may define conditions corresponding to low stress or other healthy states of being. The target health condition criteria may include defined ranges for blood pressure, body temperature, pulse rate, metabolic rates, and various other types of physiological parameters. The defined ranges may be selected to correspond to ranges known to promote healthy lifestyles.

In the example shown in FIG. 5, prompting a person to exhibit a facial expression at step 320ii also includes comparing the current health condition data to the target health condition criteria of the health condition parameter data at step 325. The comparison performed at step 325 may be used to track a user's health metrics. Additionally or alternatively, the comparison performed at step 325 may be used to trigger prompts to exhibit a facial expression.

For example, step 326 includes prompting person 102 to exhibit a facial expression satisfying the target facial expression criteria when the comparison of the current health condition data fails to satisfy the target health condition criteria. In some examples, the user is prompted to exhibit a desired facial expression immediately when the current health condition data fails to satisfy the target health condition criteria. In some examples, the user is prompted to exhibit a desired facial expression when the current health condition data fails to satisfy the target health condition criteria for a predetermined amount of time.

Returning focus to FIG. 3, system instructions 300 include receiving current facial expression data from facial expression detection device 106 at step 330. The current facial expression data may be received via a wired or wireless data connection.

At step 340, system instructions 300 include comparing the current facial expression data to the target facial expression criteria of the facial expression parameter data. After comparing the facial expression data to the target facial expression criteria at step 340, system instructions 300 include identifying whether the current facial expression data satisfies the target facial expression criteria at step 350.

In the present example, system instructions 300 include optional step 360 of determining how long the current expression data satisfied the target facial expression criteria. Other examples do not include tracking how long the target facial expression criteria was satisfied. In applications where maintaining a facial expression for a prescribed length of time is desired, such as maintaining a genuine smile for a given length of time to provide desired health benefits, tracking how long the target facial expression criteria was satisfied can assist with encouraging a user to maintain the facial expression for the prescribed time. Tracking how long the target facial expression criteria was satisfied can also help with communicating or reporting facial expression performance results.

At step 370, system instructions 300 include sending display data to display 110. The display data may communicate whether, how often, and/or for how long the target facial criteria was satisfied. The display data may also include health information, such as health benefits resulting from exhibiting facial expressions satisfying the target facial criteria.

Sending the display data to display 110 may assist the user to understand his or her facial expression performance and to make adjustments accordingly. In some examples, sending the display data to a display is performed as part of a game or contest where a user is encouraged to exhibit a desired facial expression. For example, a user may be assigned points, be awarded virtual prizes, or progress to new places in a virtual world when meeting facial expression parameters communicated to the user in the form of a game or entertainment experience.

At step 380 in FIG. 3, the reader can see that system instructions 300 include communicating results data to a social media platform, such as social media platform 122 depicted conceptually in FIG. 1. The results data may correspond to whether the current facial expression data satisfies the target facial expression criteria. Additionally or alternatively, the results data may include how often, and/or for how long the target facial criteria was satisfied. In some examples, the results data may also include health information, such as health benefits resulting from exhibiting facial expressions satisfying the target facial criteria. In examples where the system instructions incorporate or work in conjunction with a game, the results data may include the points, virtual prizes, or progress the user has achieved in the game that utilizes facial expressions.

### Display

Display 110 functions to display display data to person 102. As shown in FIG. 2, display 110 is in data communication with system processor 108. Display 110 and system processor 108 may communicate data via a wired or wireless connection.

The display may be any currently known or later developed type of display for displaying data. In the example shown in FIG. 3, display 110 is a watch screen. In the example shown in FIG. 6, display 210 is a smartphone screen. In the example shown in FIG. 7, display 410 is a laptop computer screen.

### Additional Embodiments

The discussion will now focus on additional smile detection system embodiments. The additional embodiments include many similar or identical features to smile detection system 100. Thus, for the sake of brevity, each feature of the additional embodiments below will not be redundantly explained. Rather, key distinctions between the additional embodiments and smile detection system 100 will be described in detail and the reader should reference the discussion above for features substantially similar between the different smile detection system examples.

### Second Embodiment

Turning attention to FIG. 6, a second example of a smile detection system, smile detection system 200, will now be described. As can be seen in FIG. 6, smile detection system 200 includes a facial expression detection device 206, a system processor 208, and a display 210. A distinction between smile detection system 200 and smile detection system 100 is that smile detection system 200 is incorporated into a smart phone 220 rather than into watch 120.

### Third Embodiment

Turning attention to FIG. 7, a third example of a smile detection system, smile detection system 400, will now be described. As can be seen in FIG. 7, smile detection system 400 includes a facial expression detection device 406, a system processor 408, and a display 410. A distinction between smile detection system 400 and smile detection system 100 is that smile detection system 400 is incorporated into a laptop computer 420 rather than into watch 120.

### Fourth Embodiment

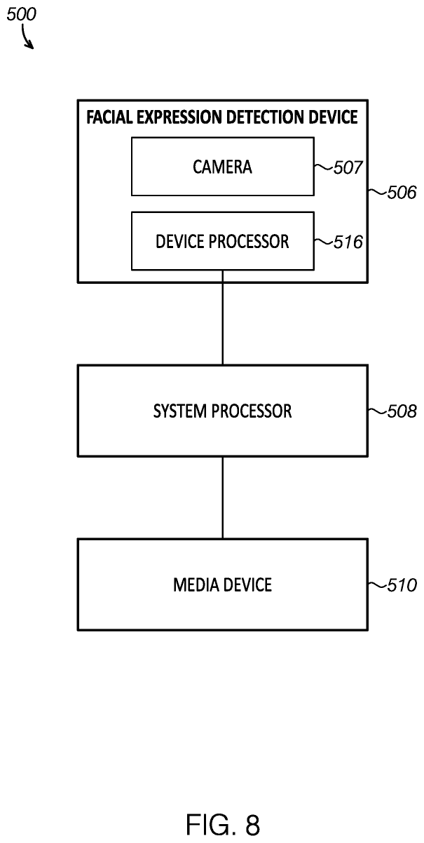

Turning attention to FIGS. 8-16, systems for controlling media playback based on a person exhibiting a smile with therapeutic benefits will now be described. As can be seen in FIG. 8, media playback system 500 includes a facial expression detection device 506, a system processor 508, and a media device 510. The components of media playback system 500 and the computer executable instructions executed by media playback system 500 are described in more detail below.

Facial expression detection device 506 is configured similarly to facial expression detection devices 106, 206, and 406 described above. As with the devices described above, facial expression detection device 506 is configured to acquire facial expression data.

As shown in FIG. 8, facial expression detection device 506 includes a camera 507 and a device processor 516. As further shown in FIG. 8, facial expression detection device 506 is controllably coupled to system processor 508. Facial expression detection device 506 may be incorporated into a handheld computing device, a watch, a television, a laptop computer, a desktop computer, and any suitable device.

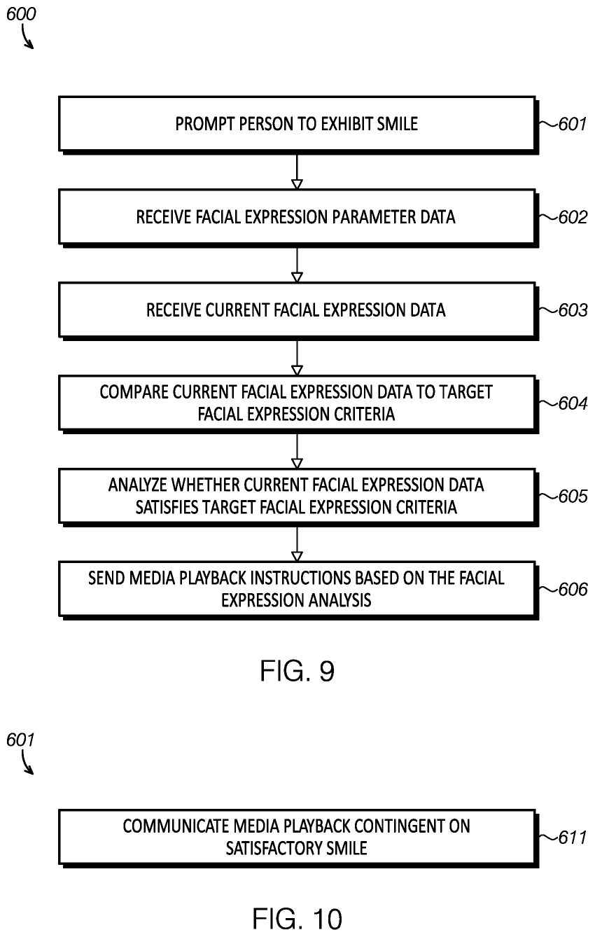

System processor 508 is configured similarly to system processors 108, 208, and 408 described above. As shown in FIG. 8, system processor 508 is controllably coupled to facial expression detection device 506 and media device 510. System processor 508 is configured to execute computer executable system instructions for establishing media playback instructions, including computer executable system instructions 600, 700, 800, and 900 shown in FIGS. 9-16 and described below.

Media device 510 is configured to play and stop playing media files in response to media playback instructions from system processor 508. In the examples discussed herein, media device 510 receives playback instructions to play and stop playing media files from system processor 508. As shown in FIG. 8, media device 510 is controllably coupled to system processor 508. Media device 510 can access media files on an external device, such as a media server, or store media files in internal memory.

### Computer Executable System Instructions

The computer executable system instructions function to encourage a person to exhibit a therapeutic smile or other therapeutic facial expression. Further, the computer executable system instructions serve to enable system processor 508 to control media playback on media device 510 based on a user's therapeutic smile performance detected by facial expression detection device 506.

### Instructions Set 1

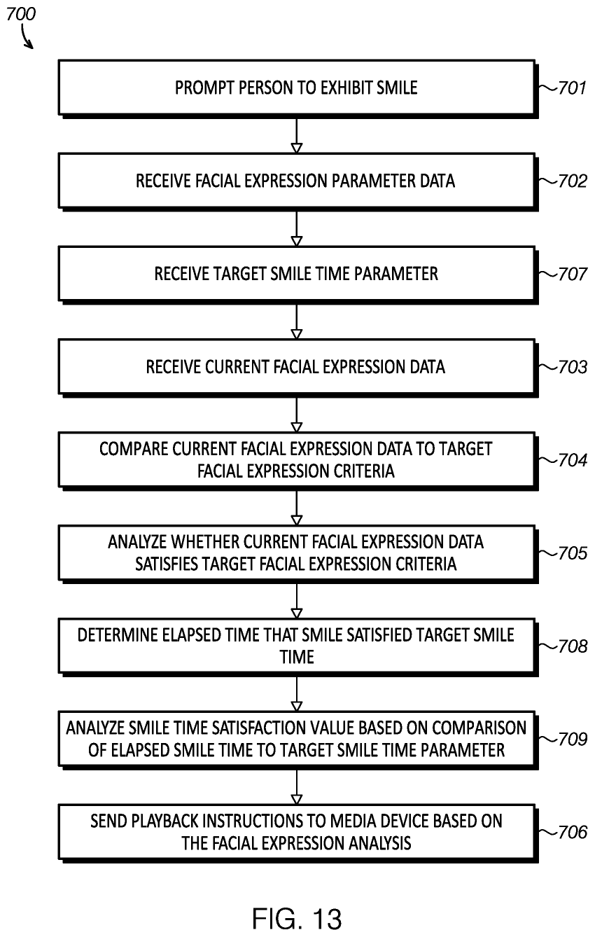

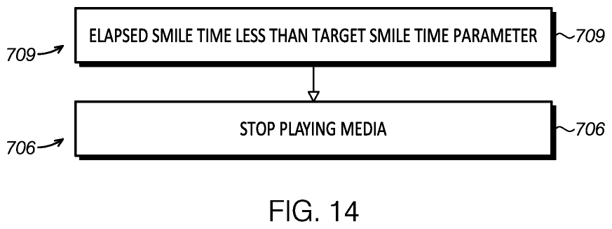

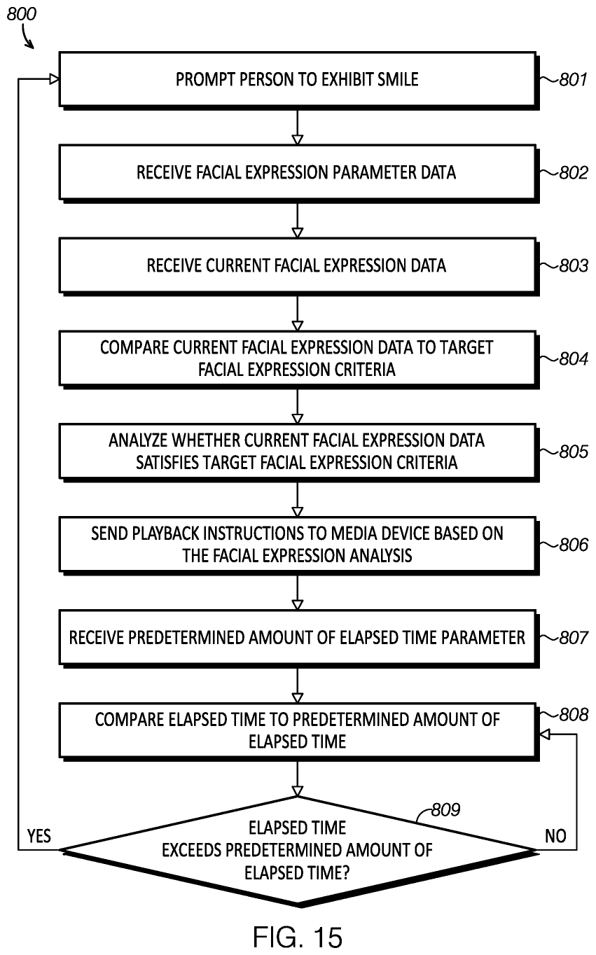

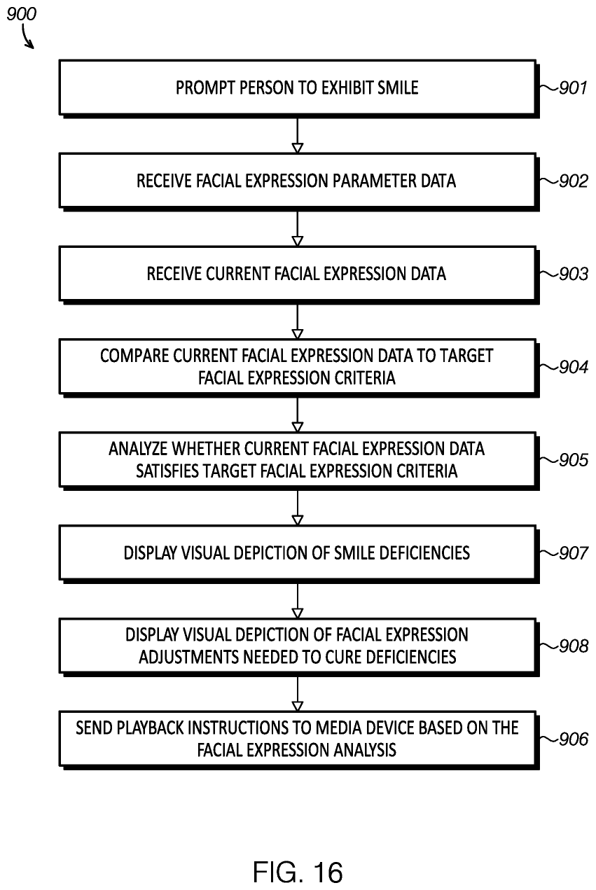

With reference to FIGS. 9-12, a first set of computer executable system instructions, system instructions 600, will be described. Additional or alternative system instructions 700 are shown in FIGS. 13 and 14 and described below. Still further additional or alternative system instructions 800 and 900 and shown in FIGS. 15 and 16, respectively, and discussed below.

### Prompting Person to Exhibit Smile

As shown in FIG. 9, system instructions 600 include prompting a person to exhibit a smile at step 601. In particular, step 601 prompts a user to exhibit a smile with therapeutic benefits. A Duchenne smile is one example of a smile with recognized therapeutic benefits. Other types of smiles or facial expressions with therapeutic benefits may be prompted in step 601 in addition or alternatively to a Duchenne smile.

As shown in FIG. 10, prompting a user to smile at step 601 may include communicating a message to the user. In the example shown in FIG. 10, prompting a user to smile at step 601 includes communicating at step 611 that media playback on media device 510 is contingent on a satisfactory smile. Media playback being contingent on a satisfactory smile may encourage a user to smile therapeutically and to thereby obtain the associated therapeutic benefits.

In some examples, communicating that media playback in contingent on a satisfactory smile at step 611 occurs before media device 510 begins playing a media file. Additionally or alternatively, communicating that media playback in contingent on a satisfactory smile at step 611 occurs while a media file is playing. For example, the message may communicate that media playback will cease if a satisfactory smile is not detected when prompted.

In some examples, instructions 600 include prompting the person to exhibit a smile with therapeutic benefits multiple times while the media device plays the media file. For example, the user may be prompted to exhibit a therapeutic smile multiple times in a therapeutic session, multiple times in a media playback session, or multiple times per media file. In other examples, the user is prompted to exhibit a therapeutic smile only once per session or once per media file. In the present example, system instructions 600 include repeating steps 602-606 each time the user is prompted to exhibit a smile at step 601.

### Receiving Facial Expression Parameter Data

Receiving facial expression parameter data at step 602 enables system 500 to evaluate whether the smile exhibited in step 601 meets criteria necessary for therapeutic benefits. The facial expression parameter data establishes target facial expression criteria defining a smile with therapeutic benefits.

The target facial expression criteria may include a variety of indicators known to be involved with therapeutic smiles or other facial expressions. For example, the target facial expression criteria may include the zygomatic major muscles of the person contracting to a selected extent. Additionally or alternatively, the target facial expression criteria may include the orbicularis oculi muscles of the person contracting to a selected extent.

### Receive Current Facial Expression Data

Receiving current facial expression data at step 603 enables system 500 to evaluate whether the user is exhibiting a therapeutic smile or other therapeutic facial expression. The current facial expression data received at step 603 corresponds to the facial expression of the person at a given time, such as when the user is prompted to exhibit a smile at step 601.

In system 500, the current facial expression data is obtained at step 603 from facial expression detection device 506. Camera 507 and device processor 516 of facial expression detection device 506 cooperate to acquire image data of a person's face, process the raw image data into current facial expression data, and to output the current facial expression data to system processor 508.

### Comparing and Analyzing Facial Expression Data

Comparing the current facial expression data to the target facial expression criteria at step 604 enables analysis of the current facial expression data in step 605. Analyzing the current facial expression data at step 605 enables system 500 to determine if the user has exhibited a therapeutic smile or other therapeutic facial expression. As shown in FIG. 9, system 500 uses the step 605 analysis to generate and send media playback instructions at step 606.

At step 604, the current facial expression data is compared to the target facial expression criteria of the facial expression parameter data received in step 603. Any currently known or later developed means of comparing the facial expression data to the target facial expression criteria may be used.

At step 605, the comparison data obtained from step 604 is analyzed to determine if the user has exhibited a therapeutic smile or other therapeutic facial expression. A wide variety of analysis methodologies may be employed, such as statistical analysis or assigning a score to the comparison data and vetting the score against a predetermined score threshold.

### Sending Media Playback Instructions

Sending playback instructions to the media device at step 606 functions to motivate a user to exhibit a therapeutic smile or other therapeutic facial expression. In the method shown in FIG. 9, system processor 508 sends media playback instructions to media device 510 at step 606 based on the analysis in step 605 regarding whether the current facial expression data satisfies the target facial expression criteria.

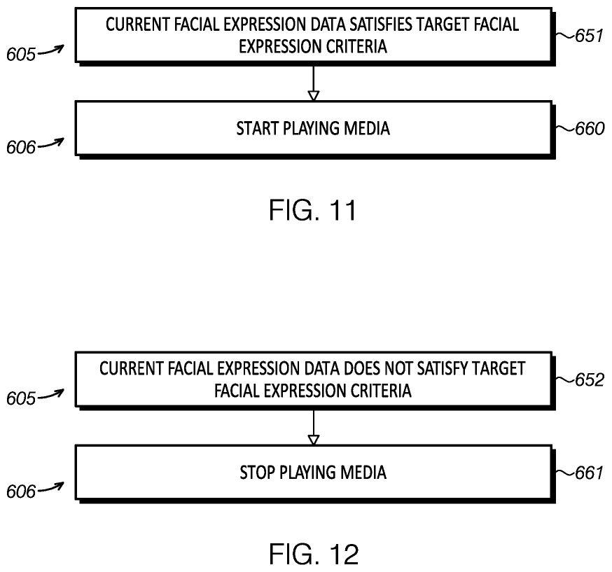

The media playback instructions at step 606 may include starting, resuming, pausing, or stopping media playback on media device 510. For example, as shown in FIG. 11, the playback instructions at step 606 include starting the media device playing the media file at step 660 when system processor 508 determines at step 652 that the current facial expression data satisfies the target facial expression criteria. As shown in FIG. 12, the playback instructions include stopping the media device playing the media file at step 661 when system processor 508 determines at step 652 that the current facial expression data does not satisfy the target facial expression criteria.

Any currently known method or program instruction paradigm may be used to control media playback on media device 510. The playback instructions at step 606 may correspond to conventional play, resume, pause, and stop functions widely used on media devices.

### Instructions Set 2

With reference to FIGS. 13 and 14, the reader can see a variation of computer executable system instructions, system instructions 700. In system instructions 700, a target smile time parameter is utilized in the analysis for media playback instructions. Steps 701-706 in system instructions 700 are substantially similar to steps 601-606 discussed above. Unique steps 707-709 are discussed below.

At step 707, system processor 508 receives a target smile time parameter. The target smile time parameter corresponds to a predetermined length of time required for the current expression data to satisfy the target facial expression criteria. The predetermined length of time may be based on medical information correlating therapeutic benefits with the amount of time a therapeutic smile is maintained. For example, if medical information indicates that maintaining a therapeutic smile for at least 10 seconds is effective to achieve therapeutic benefits, the target smile time parameter may be 10 seconds, 15 seconds, or longer.

At step 708, system processor 508 determines an elapsed smile time. The elapsed smile time corresponds to how long the current expression data received in step 703 satisfied the target facial expression criteria received in step 702. For example, the elapsed smile time would be 12 seconds if a user maintained a smile for 16 seconds, but the smile met the target facial expression criteria of a therapeutic smile for only 12 seconds.

At step 709, system processor 508 compares the elapsed smile time to the target smile time parameter. Comparing the elapsed smile time to the target smile time parameter yields a smile time satisfaction value. The smile time satisfaction value corresponds to whether the elapsed smile time is less than, greater than, or equal to the target smile time parameter.

In some examples, the smile time satisfaction value is a binary true/false or pass/fail value. For example, the smile time satisfaction value may be true or pass if the elapsed smile time is greater than or equal to the target smile time parameter. Correspondingly, the smile time satisfaction value may be false or fail if the elapsed smile time is less than the target smile time parameter.

The reader can see in FIGS. 13 and 14 that system processor 508 sends playback instructions at step 706 to media device 510 based on the smile time satisfaction value. For example, at step 761 in FIG. 14, system processor 508 stops media playback at step 761 when it determines at step 790 that the elapsed smile time is less than the target smile time parameter. In other examples, the system processor starts or resumes media playback when the smile time satisfaction value indicates that the elapsed smile time is greater than or equal to the target smile time parameter.

### Instructions Set 3

With reference to FIG. 15, the reader can see a variation of computer executable system instructions, system instructions 800. In system instructions 800, a user is prompted to exhibit a facial expression satisfying the target facial expression criteria after a predetermined amount of elapsed time. Steps 801-806 in system instructions 800 are substantially similar to steps 601-606 and 701-706 discussed above. Unique steps 807-809 are discussed below.

At step 807, a predetermined amount of elapsed time parameter is received. The predetermined amount of elapsed time parameter establishes when the user will next be prompted to exhibit a smile with therapeutic benefits at step 801 of system instructions 800. In some examples, the predetermined amount of elapsed time is a regular time interval based on a set schedule, such as every 10 minutes. In certain examples, the predetermined amount of elapsed time is a time interval based on when the person last exhibited a facial expression satisfying the target facial expression criteria. For example, the predetermined amount of elapsed time may be 15 minutes since a person last exhibited a therapeutic smile.

At step 808, the system compares how much time has elapsed since a predetermined trigger event to the predetermined amount of elapsed time parameter. The predetermined trigger may be when the user was last prompted to exhibit a smile at step 801 regardless whether the smile exhibited was determined to satisfy target facial expression criteria at step 805. Alternatively, the predetermined trigger may be when the analysis at step 805 last determined that the smile exhibited by the user satisfied the target facial expression criteria at 805.

The reader can see in FIG. 15 a decision point 809 where the system acts on the time comparison in step 808. At decision point 809, instructions 800 repeat by prompting a person to exhibit a smile at step 801 if the time elapsed from a predetermined trigger event exceeds the predetermined elapsed time parameter at step 808. If the time elapsed from a predetermined trigger event does not yet exceed the predetermined elapsed time parameter at step 808, then the instructions return to the time comparison step 808.

### Instructions Set 4

With reference to FIG. 16, the reader can see a variation of computer executable system instructions, system instructions 900. In system instructions 900, system processor 508 communicates deficiencies in a user's smile and communicates instructions for adjusting the user's smile to achieve therapeutic benefits. In particular, system processor 508 utilizes system instructions 900 to assist a user to visually identify when a smile exhibited does not have sufficient therapeutic benefits. Further, system processor 508 utilizes system instructions 900 to visually guide a user to adjust the smile to achieve increased therapeutic benefits.

Steps 901-906 in system instructions 900 are substantially like steps 601-606, 701-706, and 801-806 discussed above. Unique steps 907 and 908 are discussed below.

At step 907, system processor 508 displays a visual depiction of smile deficiencies when appropriate. For example, system processor 508 displays a visual depiction of smile deficiencies when it determines at step 905 that the current facial expression data received in step 903 does not adequately compare at step 904 to facial expression parameter data received at step 902. Additionally or alternatively, the system processor may display a visual depiction of smile deficiencies whenever there are areas where the smile could be improved. For example, a user's current smile may adequately meet the target facial expression parameter data, but could have even more therapeutic benefits if modified in one or more ways depicted visually by the system processor.

The visual depictions may take a wide variety of forms. For example, an image of a generic or simplified face may be displayed with animations or overlays indicating facial expression issues. For instance, an arrow, a red translucent overlay, or flashing symbols may be displayed next to the corners of a person's lips if the corners of the lips are not lifted sufficiently high to have therapeutic benefits. In some examples, an image of the user's actual face may be displayed with indicator symbols or features indicating where smile deficiency issues are present.

At step 908, system processor 508 communicates to the user instructions for adjusting the current facial expression. In particular, system processor 508 communicates instructions to adjust the current facial expression to satisfy the target facial expression criteria. In the example shown in FIG. 16, system processor 508 displays a visual depiction of facial expression adjustments effective to achieve therapeutic benefits at step 908.

A variety of visual depictions to facilitate adjustments may be displayed. For example, an image of a generic or simplified face may be displayed with animations or overlays indicating facial expression adjustments to make. For instance, an arrow, a series of dots or curves depicting motion, or flashing symbols may be displayed above eyebrows on the face depicted to indicate that eyebrows should be raised.

In some examples, an image of the user's actual face may be displayed with symbols or other indicators indicating where smile adjustments should be made. For instance, the adjustment indicator may include a simulated depiction of the user's lips smiling wider or higher. Artificial intelligence video generation techniques may be utilized to simulate the user's own features adjusted in selected ways.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A system for controlling media playback based on a person exhibiting a smile with therapeutic benefits, comprising:
a facial expression detection device configured to acquire facial expression data;
a system processor in data communication with the facial expression detection device and configured to execute stored computer executable system instructions;
a media device controllably coupled to the system processor and configured to play and stop playing a media file in response to playback instructions from the system processor;
wherein the computer executable system instructions include the steps of:
prompting the person to exhibit a smile with therapeutic benefits;
receiving facial expression parameter data establishing target facial expression criteria defining a smile with therapeutic benefits;
receiving current facial expression data corresponding to the facial expression of the person from the facial expression detection device;
comparing the current facial expression data to the target facial expression criteria of the facial expression parameter data;
identifying whether the current facial expression data satisfies the target facial expression criteria; and
sending playback instructions to the media device based on the identification whether the current facial expression data satisfies the target facial expression criteria.

2. The system of claim 1, wherein the playback instructions start the media device playing the media file when the current facial expression data satisfies the target facial expression criteria.

3. The system of claim 1, wherein the playback instructions stop the media device playing the media file when the current facial expression data does not satisfy the target facial expression criteria.

4. The system of claim 1, wherein the computer executable system instructions further comprise determining an elapsed smile time corresponding to how long the current expression data satisfied the target facial expression criteria.

5. The system of claim 4, wherein the computer executable system instructions further comprise:
receiving a target smile time parameter corresponding to a predetermined length of time required for the current expression data to satisfy the target facial expression criteria;
comparing the elapsed smile time to the target smile time parameter to yield a smile time satisfaction value corresponding to whether the elapsed smile time is less than, greater than, or equal to the target smile time parameter; and
sending playback instructions to the media device based on smile time satisfaction value.

6. The system of claim 5, wherein the playback instructions stop the media device playing the media file when the smile time satisfaction value indicates that the elapsed smile time is less than the target smile time parameter.

7. The system of claim 1, wherein the target facial expression criteria includes:
the zygomatic major muscles of the person contracting to a selected extent; and
the orbicularis oculi muscles of the person contracting to a selected extent.

8. The system of claim 1, wherein the facial expression detection device includes a camera.

9. The system of claim 1, wherein the facial expression detection device is incorporated into a handheld computing device.

10. The system of claim 1, wherein the facial expression detection device is incorporated into a watch.

11. The system of claim 1, wherein the computer executable system instructions further comprise prompting the person to exhibit a smile with therapeutic benefits multiple times while the media device plays the media file.

12. The system of claim 11, wherein the computer executable system instructions further comprise repeating the following steps each time the person is prompted to exhibit a smile with therapeutic benefits:
receiving current facial expression data corresponding to the facial expression of the person from the facial expression detection device;
comparing the current facial expression data to the target facial expression criteria of the facial expression parameter data; and
identifying whether the current facial expression data satisfies the target facial expression criteria; and
sending playback instructions to the media device based on the identification whether the current facial expression data satisfies the target facial expression criteria.

13. The system of claim 1, wherein the computer executable system instructions further comprise prompting the person to exhibit a facial expression satisfying the target facial expression criteria after a predetermined amount of elapsed time.

14. The system of claim 13, wherein the predetermined amount of elapsed time is a regular time interval based on a set schedule.

15. The system of claim 13, wherein the predetermined amount of elapsed time is a time interval based on when the person last exhibited a facial expression satisfying the target facial expression criteria.

16. The system of claim 1, wherein prompting the person to exhibit a smile with therapeutic benefits includes communicating to the person that the media device will stop playing the media file if the person does not exhibit a smile with therapeutic benefits that satisfies the target facial expression criteria.

17. The system of claim 16, wherein the computer executable system instructions further comprise communicating to the person deficiencies in the current facial expression data compared to the target facial expression criteria.

18. The system of claim 17, wherein communicating to the person deficiencies in the current facial expression data includes displaying a visual depiction of the deficiencies.

19. The system of claim 17, wherein the computer executable system instructions further comprise communicating to the person instructions for adjusting the facial expression to satisfy the target facial expression criteria.

20. The system of claim 19, wherein communicating to the person instructions for adjusting the facial expression includes displaying a visual depiction of the facial expression adjustments.
